(19) United States
(12) Patent Application Publication
(10) Pub. No.: US 2022/0412939 A1
(43) Pub. Date: Dec. 29, 2022

(54) SYSTEM AND METHOD FOR DETECTION AND DISPOSAL OF MICROORGANISMS AND DETECTION MODULE DISPOSED IN A WATER FLOW POINT

(71) Applicants: SOCIEDADE BENEFICENTE ISRAELITA BRASILEIRA HOSPITAL ALBERT EINSTEIN, São Paulo (BR); I-HEALTHSYS PRODUTOS MÉDICOS LTDA - ME, São Carlos (BR)

(72) Inventors: ALEXANDRE RODRIGUES MARRA, São Paulo (BR); MARCELO PRADO, São Carlos (BR); RENALDO MASSINI JUNIOR, São Carlos (BR)

(21) Appl. No.: 17/897,711

(22) Filed: Aug. 29, 2022

Related U.S. Application Data
(62) Division of application No. 16/923,634, filed on Jul. 8, 2020.

(30) Foreign Application Priority Data
Jul. 8, 2019 (BR) 10 2019 014126 3

(51) Int. Cl. G01N 33/18; C12M 1/00

(52) U.S. Cl. CPC G01N 33/1866; G01N 33/1893; C12M 1/00

(57) ABSTRACT

A system and method for the detection and elimination of microorganisms in a water flow is provided. The method includes arranging at least one light emission element at a water flow point, arranging of at least one light capture element at the water flow point, detecting the presence of the microorganism through the first light emission event and eliminating the microorganism through the realization of a second light emission event. A detection module is also provided which can be positioned at a water flow point.

### CROSS-REFERENCE TO RELATED APPLICATIONS

This application is a Divisional application of U.S. application Ser. No. 16/923,634, filed Jul. 8, 2020, which claims priority to Brazilian Patent Application No.: BR 10 2019 014126 3, filed Jul. 8, 2019, all of said applications being incorporated herein by reference.

### FIELD OF THE INVENTION

This invention concerns a system and method for the detection and elimination of microorganisms. More specifically, this invention concerns a system and method capable of detecting the presence of biofilms and/or bacteria located at a water flow point.

### DESCRIPTION OF THE STATE OF THE ART

In hospital environments, there is a constant search for procedures intended to reduce the proliferation of viruses and bacteria and, consequently, to avoid the occurrence of infections.

More specifically, it is common to seek ways to prevent or reduce the proliferation of bacteria transmitted by inhalation, that is, through breathing air that may be contaminated by a given bacterium (microorganism).

In a non-limiting exemplification, hospital environments have increasingly invested in means of avoiding transmission and contamination by the bacterium known as Legionella (Legionella pneumophila).

As we know, Legionella is a bacterium capable of causing very serious effects in human beings, such as severe pneumonia in conjunction with respiratory failure. Capable of affecting any person, Legionella preferentially targets immunocompromised patients (diabetics, the elderly, transplant patients, among others), due to the vulnerability of the defense systems of such people.

Its natural habitat comprising reservoirs and water flows, such as piping (preferably locating itself in biofilms, it is common for Legionella to proliferate in the water pipes of old buildings) rivers, lakes, taps, among others, Legionella infection occurs by air, through the inhalation of water droplets that are contaminated with the bacteria and that are generated, for example, when turning on a hot water shower/tap.

As a challenge for those who seek an effective method for the detection of this bacterium, its positive detection in water samples has always been very low; additionally, this situation is hampered by the fact that there is no standardized approach to the detection of the bacteria in water samples, let alone water samples found in hospital environments.

One way of detecting the microorganism is through the collection of large volumes of water, such as 100 ml (milliliters), 250 ml, 500 ml and up to 1000 ml, from each water point. Additionally, in some situations it is necessary to collect a high number of water samples. In this regard, we can cite the publication “Controlling Legionella pneumophila in water systems at reduced hot water temperatures with copper and silver ionization”, which can be accessed via the link https://www.sciencedirect.com/science/article/pii/S0196655318311490, where over 1500 water samples were collected.

In addition, it is recommended to collect water at different temperatures (cold and hot) from all water outlet points, which obviously ends up becoming a laborious process.

Moreover, said collection of water generally only occurs when there is a suspected or confirmed case within the hospital environment, which is to say, the action is corrective, not preventive, as it should be.

Another detail that explains the low positive rate of detection of this microorganism is the fact that, in addition to being an intracellular pathogen (it needs a cell to develop), Legionella is a bacterium that is hosted in free-living amoebas, which fact hinders the diagnosis and eradication of this pathogen in water samples.

The state-of-the-art reveals ways to detect microorganisms in water samples, as discussed in document U.S. Pat. No. 9,206,461. In this priority, a sensor for the detection of microorganisms (bacteria) is described based on the variation in the resonance frequency of a crystal oscillator.

Moreover, this document proposes the use of a polymeric layer to detect the shape of the microorganism more specifically; basically, the microorganisms are attracted by potential difference (electrical/static charge) to the polymer, so that it forms a kind of “mold” of the microorganism. Once the bacteria is detected, it is then destroyed, leaving only the mold of the microorganism in the polymer.

One of the disadvantages of this form of detection it that with each new measurement (or at short intervals), the polymer in question must be changed. This fact hinders and prevents the use of the methodology in question in the water pipes of large buildings, such as hospital environments.

The priority US 2018/0195035 also describes a methodology and a device capable of isolating and detecting pathogens in water samples. Basically, this document addresses ways of detecting pathogens in water using, as a basis, the binding of the pathogens to a given resin, where said binding is caused by electrostatic interactions.

The device described in this priority comprises two fluidly connected portions, where the water under analysis must be inserted through the first portion and moves to the second portion. It is also proposed that the second portion should comprise the retaining resin, capable of allowing the passage of liquid but also capable of blocking the passage of certain particles.

Analyzing the description of the steps necessary for the use of the device proposed in document US 2018/0195035, it is observed that these steps basically consist of steps to be performed in laboratories, that is, it is probably impossible to use the proposed device for continuous monitoring, in real time, of a flow of water that flows through a given hydraulic pipe, such as the piping of a hospital environment.

Thus, there is a gap in the state of the art relating to the proposal of a system and method capable of detecting the existence of microorganisms in water flows, where said detection occurs in real time and continuously, which is to say, said system and methodology are capable of being internally located at a water flow point (pipe), thus allowing for the continuous evaluation of the water to determine whether it contains a given microorganism.

The present invention fills the gap in the state of the art by proposing a system and method that are based on the use of a light emission element to be positioned at a water flow point, thus allowing for the detection of microorganisms.

Moreover, the teachings of the present invention make it possible, when a sample of contaminated water is detected, for the same system used to detect the bacterium to be used to eliminate the microorganism, as shall be detailed below.

Among the advantages of the methodology and system hereby proposed, we may cite: (i) the identification of water samples in real time and continuously, (ii) the lack of need to handle large volumes of water (100 ml to 1 liter per water point, according to the state of the art), since no handling is required for the collection of water and its sending to the laboratory, consequently producing (iii) savings in terms of lab work and the (iv) possibility of eradicating the bacterium by various methods and the analysis of which method is the most effective.

In a non-limiting example, Legionella eradication could occur by increasing water temperature or chlorination, releasing silver and copper ions into the water, releasing monochloramine into the water and ultraviolet emission. It is worth noting that such methods could be used in isolation or together, such as combining the increase in temperature with flushing (increasing the water speed) and increased chlorination. Obviously, this description should not be considered as a limiting feature of the present invention.

Moreover, the teachings of the present invention allow for (iv) the transition from purely corrective actions to preventive actions, (v) the possibility that the point of detection of the bacterium is traced, thus making it possible to know at which location of the pipe the microorganism was detected and (vi) the protection of the environment and the patients of the hospital unit.

In the light of the foregoing, and based on the above description, the detection and control of Legionella is a challenge for the medical sector. This is because prevention has been attempted, but ineffectively, precisely due to the difficulty of the microbiological methods (in detecting the bacteria in water), and the need to collect huge amounts of water, where no microorganism (bacterium) is generally detected.

Even using molecular biology methods to detect Legionella has not been successful in improving diagnosis, since it involves an intracellular pathogen and the presence of multiple species.

Some methodologies use genomic sequencing, which allows for the verification of the existence of several species of the bacterium. In any case, to perform genomic sequencing, one must isolate/identify the bacterium first. An additional difficulty lies in the fact that there is a difference between detecting Legionella in water and detecting Legionella in the patient. Often the bacterium is found in the patient but not in the water samples, when it is known that the probability that the water sample contains the bacteria is immense.

Through genomic sequencing studies, it has been verified that the same species of Legionella may remain in the water pipes of hospitals for periods of more than 30 years.

Thus, there is a need, in the state of the art, for a system and methods capable of detecting the presence of microorganisms in water flows, so that this detection can occur in real time, allowing for the preventive monitoring of water pipes to be performed.

As such, a system and method for the detection and elimination of microorganisms in a water flow is described, where the term ‘microorganisms’ is understood to mean at least one of the following: a biofilm, a biofilm that hosts a given bacterium and a bacterium.

### SUMMARY OF THE INVENTION

The present invention is intended to provide a system and method capable of detecting the presence of a microorganism at a water flow point.

An additional aim of the present invention is to enable the detection of the microorganism to occur in real time, thus indicating to a user of the system that a certain water flow point contains the detected microorganism.

It is also an aim of the present invention to enable the proposed system to be positioned inside a water pipe, allowing said system to be moved along the water flow.

The present invention also aims to provide a methodology and system that uses a light emitting element and a light capture element to detect a microorganism at a water flow point.

The present invention also aims to provide a methodology and system that uses a crystal element, such as a quartz crystal sensor, to detect a microorganism at a water flow point.

It is an additional aim of the present invention to provide a methodology and system that uses a crystal element (such as a quartz crystal sensor) together with a light emitting and capture element to thus detect the presence of a microorganism at a water flow point.

The present invention also aims to provide a methodology and system where the light emitting element is concentrically arranged along the water flow point.

An additional goal of the present invention resides in a methodology and system capable of eradicating the microorganism from the water flow point.

The present invention also aims to provide a methodology and system to be used in a hospital environment.

### BRIEF DESCRIPTION OF THE INVENTION

The objectives of the present invention are achieved initially by a method for the detection and elimination of microorganisms in a water flow. More specifically, the method comprises the steps of: arranging at least one light emitting element at a water flow point, arranging at least one light capture element at the water flow point and detecting the presence of the microorganism through the performance of a first light emission event, where the method also comprises the stage of eliminating the microorganism through the performance of a second light emission event.

More specifically, the first light emission event also comprises the steps of: emitting a first beam of light at a target point and evaluating the behavior of the target point in response to the first beam of light emitted. More specifically, the first beam of light is intended to excite the bioluminescence of the target point T, thus allowing for the capture of this intensity of emitted light.

Furthermore, the second light emission event comprises the steps of: emitting a second beam of light at the target point if the microorganism has been detected and eliminating the microorganism through the emission of the second beam of light, where the second beam of light is configured as a laser beam.

Generally speaking, the emission power of the first beam of light is less than the emission power of the second beam of light. Additionally, from the teachings proposed in the present invention it is understood that the first beam of light is used for the detection of the microorganism and the second beam of light is used for the destruction of the microorganism.

### BRIEF DESCRIPTION OF THE DRAWINGS

FIG. 1—is a representation of the system for the detection and elimination of microorganisms proposed in the present invention, indicating the positioning of a light emission element at a water flow point.

FIG. 2—is a representation of the system for the detection and elimination of microorganisms proposed in the present invention, indicating the realization of a first light emission event;

FIG. 3—is a representation of the system for the detection and elimination of microorganisms proposed in the present invention, indicating the realization of a second light emission event;

FIG. 4—is a graphic representation of emission possibilities of the first beam of light and/or the second beam of light, wherein FIG. 4(a) represents the emission in a pulsed manner, FIG. 4(b) represents the emission continuously and FIG. 4 (c) illustrates the emission combining the pulsed and continuous form.

FIG. 5—is a representation of the system for the detection and elimination of microorganisms proposed in the present invention, indicating the positioning of a detection module at the water flow point;

FIG. 6—is a representation of the system for the detection and elimination of microorganisms proposed in the present invention, indicating the positioning of a detection module at the water flow point, where said water flow point is configured as a tap;

FIG. 7—illustrates an additional representation of the detection module, wherein FIG. 7 (a) represents a side view of said module and FIG. 7 (b) illustrates an upper view of said element.

FIG. 8—illustrates an additional representation of the detection module, wherein FIG. 8 (a) represents a front view of said module and FIG. 8 (b) illustrates a side view of said element;

FIG. 9—is a representation of the system for the detection and elimination of microorganisms proposed in the present invention, where said system comprises the detection module associated with the light emission element.

### DETAILED DESCRIPTION OF THE FIGURES

The teachings of the present invention concern a system and method for the detection and elimination of microorganisms.

More specifically, the present invention proposes a system and method capable of detecting the existence of a microorganism in a water flow.

In relation to the term water flow, and considering this configuration of the present invention, this can be understood as the volume of water moving along a pipe 6, said displacement occurring from a starting point to an end point.

Thus, said pipe 6 can represent the plumbing of a given location, said plumbing 6 being responsible for capturing water from an entry point to a given exit point.

In one configuration, the entry point can be understood as the point at which the flow of water supplied by a responsible company is delivered to a particular location (such as a house, for example), while the exit points represent the water outlet points of the site in question. Thus, in one configuration, the outlet points can be coupled to showers, faucets, drinking fountains, toilets, among others.

The displacement of the water flow along a given pipe 6 (pipeline) is not a limiting feature of the present invention, such that the flow of water need not necessarily move from one point to another.

Specifically, the term water flow can also be understood as the volume of water that is stored, dammed and/or positioned at a given location. Thus, the teachings of the present invention can be perfectly applied to water tanks, lakes, machine tanks, toilet tanks, drinking fountains, refrigeration and air conditioning units, among others.

Generally speaking, any place that contains water (whether in motion or not), at any temperature, would be able to incorporate the teachings of the present invention.

For a better understanding of the present invention, the term water flow shall be described as referring to the volume of water moving in a given pipe (duct/pipeline).

In one non-limiting configuration, said water flow can be part of a hospital environment, such that, in this configuration of the present invention, the term hospital environment can be understood as a hospital.

Hospital environment can also be understood to mean any place used to accommodate a given patient, regardless of the time period or reason for accommodation (surgery, rest, treatment, among others).

It is worth noting that the term hospital environment need not necessarily refer to a hospital, such that any health unit, such as clinics, doctors' offices, wards, surgical centers and emergency care units can also be understood as hospital environments.

In general terms, and in order to better understand the invention, the term hospital environment can be understood as any place used to accommodate a given patient, whether for long or short periods of time.

Thus, the interpretation of the term hospital environment as a hospital does not represent a limitation of the present invention. Nor does the use of the concepts proposed herein in a hospital environment represent a limitation of the present invention, such that the water flow referred to in the present invention could be located at any site, such as buildings or houses.

In this configuration of the present invention, the proposed system and method are used to detect the presence of a given microorganism in the water flow.

By microorganism, one may understand, for example, a biofilm, a certain bacterium (such as Legionella) as well as the combination of biofilm and bacteria. It is worth noting that the reference to Legionella should not be considered as a limiting characteristic of the present invention. In general terms, the teachings proposed herein can be used in the detection of any microorganism capable of proliferating in water.

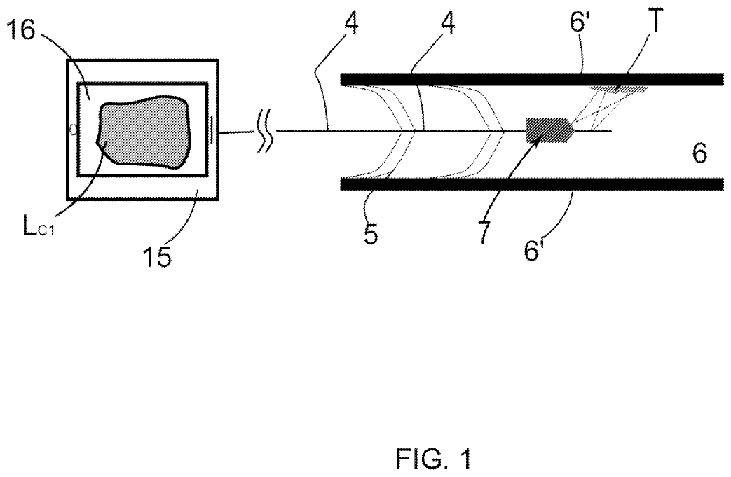

The present invention initially proposes a method for the detection and elimination of microorganisms in a water flow, such as for the detection of biofilms found in a water flow. In reference to FIG. 1 and as discussed earlier, it is understood that the water flow moves in a pipe 6 located in a hospital environment, thus said piping 6 is delimited by an external wall 6′, as illustrated in FIG. 1.

It is worth noting that the structural configuration of the cross-section of the piping 6 is irrelevant considering the teachings of the present invention.

For the detection of the microorganism, the methodology proposed hereby uses a light emission element 4 to be positioned at a water flow point 6. In other words, it is understood that said light emission element 4 must be positioned in the inner portion of the piping 6, as represented in FIG. 1. In this configuration of the invention, the use of the light emission element 4 proved to be extremely effective in the detection and elimination of biofilms located in the piping 6, so that such biofilms tend to comprise a plurality of bacteria located on its surface.

Specifically, the light emission element 4 should be understood as an element capable of emitting a given light intensity inside the piping 6. So, in this configuration of the present invention, it is proposed that the light emission element 4 be configured as a fiber optic, such as a side-fire fiber optic (fiber with sidelight emission).

As is known, fiber optics usually transmit light in the direction of the fiber itself, that is, in the direction of the longitudinal axis of the fiber optic. In this regard, the side fire fiber optic is configured to emit light at an angle approximately perpendicular to the longitudinal axis of the fiber, which is to say, the light is emitted laterally in relation to the fiber, thus allowing the external wall 6′ (the inner surface of said wall) of the pipe to be struck by the emitted light.

Obviously, the proposed use of a side fire fiber optic should not be considered a limitation of the present invention, such that any element capable of emitting light in the direction of the outer wall 6′ may be used.

In one configuration, the light emission element 4 must be concentrically arranged along the water flow point 6. Thus, it is understood that said element must be concentrically arranged along the water pipe 6.

To this end, it is proposed that at least one elastic element 5 be coupled to the fiber optic and also to the outer wall 6, thus allowing for the correct positioning of the fiber inside the pipe 6, and more specifically in the center of the pipe.

In one valid but non-limiting configuration, said elastic element 5 may be configured as a spring 5, or a plurality of springs 5 associated with the fiber at one end and the outer wall 6 at the opposite end, as illustrated in FIG. 1.

It is worth noting that the proposed configuration of the elastic element as a spring 5 does not represent a limiting characteristic of the present invention, such that any element that acts as a support of the light emission element 4 and that allows for its introduction and positioning in the central portion of the piping 6 may be used.

In addition to the use of the elastic element 5, the light emission element 4 may be positioned (encapsulated) inside a tubular structure (casing), where only the tip of the fiber (the region encompassing the light source) is positioned outside said structure. Furthermore, this structure should allow for its movement along the pipe 6. In other words, it will be possible for the structure to be pushed and/or pulled along the pipe 6 and through an access point, thus allowing for its positioning at the point of interest.

It is proposed that this structure have sufficient lateral flexibility to allow for its movement along the pipe 6 and thus pass through any curved points. In addition, said structure must have sufficient longitudinal rigidity to allow it to be “pushed/pulled” along the pipe 6 without the structure being deformed longitudinally.

In summary, it is understood that the positioning of the light emission element 4 inside the tubular structure uses concepts derived from the engineering device known as the borescope.

Thus, one can combine the use of the elastic element 5 with the arrangement of the fiber 4 within said structure, thus allowing for the positioning and movement of the fiber along the piping 6 in addition to allowing for its use in pipes of different diameters.

It is also worth noting that the length of said structure and/or fiber optic 4 must fulfill the purpose of its use. In other words, the length of the structure/fiber must be consistent with the desired application, such as the inspection of pipes and piping as well as the inspection of water tanks. Generally speaking, the length of the structure and/or fiber need not represent a limiting characteristic of the present invention.

The method and system for the detection and elimination of microorganisms also comprises the step of positioning at least one light capture element 7 at the water flow point 6.

More specifically, and in reference to FIG. 1, the light capture element 7 should be understood as a micro camera 7 associated with the fiber optic and preferably also arranged concentrically in relation to the pipe 6. If the fiber optic is surrounded by the previously described structure, it is understood that the light capture element 7 must be associated with said structure. It is also proposed that the element 7 comprises a protective housing, thus allowing for its use in wet environments. The state of the art already reveals a plurality of means of protection for cameras, thus allowing for the use thereof in water.

Generally speaking, it is proposed that the light capture element 7 be able to have its focus on the outer wall 6′, and more specifically on the region where the fiber 4 directs its beam of light.

Thus, and based on the use of the fiber 4 in conjunction with the light capture element 7, the presence of the microorganism in the pipe 6 can be detected.

More specifically, the use of the fiber optic 4 in conjunction with the light capture element 7 will allow for the detection of the presence of a biofilm in the pipe 6. In this regard, it is known that microorganisms (such as Legionella) are found in biofilms, such that these microorganisms also contain amino acids.

One of the amino acids present in microorganisms is tryptophan, which possesses the characteristic of emitting fluorescent light when being irradiated/struck by a beam of light of a certain wavelength. Thus, the present invention proposes that the light emitted by the amino acid can be detected by the light capture element 7, thus allowing for the detection of the existence of the biofilm inside the pipe 6.

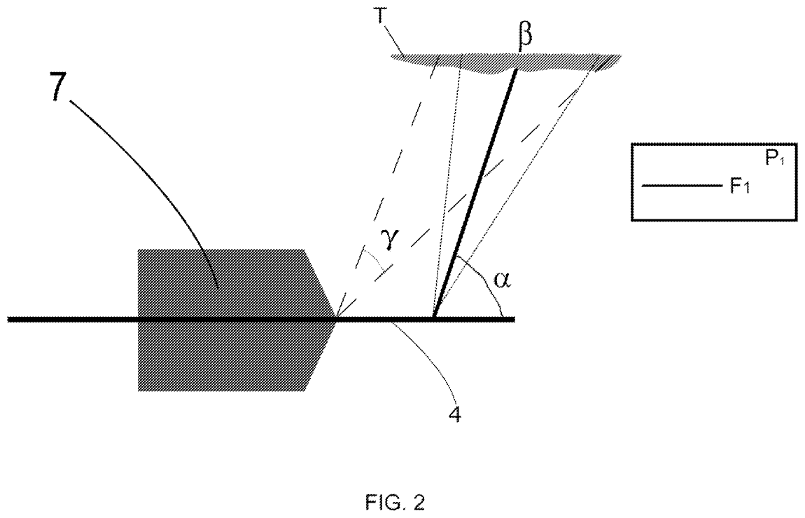

Thus, and in reference to FIGS. 1 and 2, the teachings of the present invention propose that the microorganism be detected through the realization a first light emission event P₁. More specifically, the first light emission event P₁ comprises the steps of emitting a first beam of light F₁ at a target point T and evaluating the behavior of the target point T in response to the first beam of light F₁ emitted.

In this configuration, the beam of light F₁ is emitted at the wavelength of the ultraviolet, which is to say, between 200 to 400 nm (nanometers), which fact ensures that the target point T, when struck by this radiation, will display a certain behavior and thus enable the detection of the biofilm that may be positioned inside the pipe 6 and consequently at the target point T.

In a purely illustrative description, the first beam of light F₁ is emitted steadily at 250 nm, and, in a similarly illustrative manner, the first beam F₁ should be emitted for a time period 10 seconds. It is worth noting that the reference to this time period should not be considered as a limiting characteristic of the present invention.

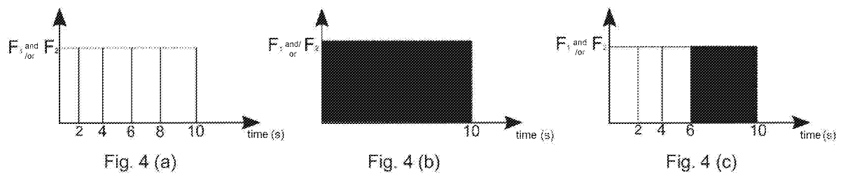

In emitting the first beam of light F1 in a constant (continuous) manner, it is understood that the first beam is emitted uninterruptedly for the desired period of time, as shown in FIG. 4 (b).

In relation to target point T, this should be understood as the region of the piping 6 where the presence of the microorganism is to be evaluated, in other words, the target point T can be understood as a biofilm (containing a bacterium) that is housed in the piping 6, and more specifically in its side wall 6′, as indicated in FIGS. 1, 2 and 3.

Thus, when irradiated by the first beam of light F₁, the biofilm will display bioluminescent behavior, which is to say, the biofilm will emit a certain intensity of light, so that said bioluminescence of the target point T should be captured by the camera 7 positioned in the pipe 6.

In other words, it is understood that the teachings proposed in the present invention determine that the instant the first beam of light F₁ strikes the target point T, the light capture element 7 will measure the intensity of the bioluminescence of the target point L₁. Thus, a synchronization is proposed between the emission of the first beam of light F₁ by the fiber 4 and the capture of bioluminescence L₁ by the camera 7. With a view to providing a better understanding of this description, the bioluminescence of the target point T detected after the emission of the first beam of light is also referred to as initial bioluminescence L₁.

In other words, it is proposed that the capture of the initial bioluminescence L₁ by the light capture element 7 occurs at a time immediately after the application of the first beam of light F₁, such that the term ‘immediately after’ hereby means a period of time not exceeding 150 milliseconds, so that a range between 50 ms and 150 ms is fully acceptable. Thus, considering that the first beam F₁ was applied at an instant t=0 second, the capture of the initial bioluminescence L₁ should occur between 50 ms and 150 ms.

FIG. 2 illustrates a detail regarding the realization of the first light emission event P₁, as previously described. The application of the first beam F₁ (solid line) from the light emission element 4 and towards the target point T is observed, thus describing a first angle α in relation to the longitudinal axis of the fiber 4 as well as a capture amplitude β (angle of divergence) on the surface of the target point T.

After the application of the first beam F₁, the light capture element 7 is triggered with an opening amplitude indicated by the Greek letter γ, so that the aperture amplitude γ should encompass the capture amplitude β of the fiber 4, as illustrated in FIG. 2, thus allowing for the detection of the bioluminescence intensity emitted by the target point T.

The evaluation of the intensity of the initial bioluminescence of the target point L₁ consists of the evaluation of the behavior of the target point T in relation to the first beam F₁ emitted, i.e., the evaluation of the intensity of the initial bioluminescence L₁ will allow for the evaluation of whether or not the target point T comprises a particular biofilm or a particular microorganism housed in the biofilm, such as Legionella. In one fully valid modality of the present invention, the intensity of the bioluminescence emitted by the biofilm (referred to as the range of action) is located in the range of 300 nm to 380 nm.

More specifically, and for this evaluation to be able to occur, a microprocessor 15 must be coupled to both the fiber 4 and the capture element 7. The mode of association of the microprocessor 15 with the fiber 4 and the capture element 7 does not represent an essential characteristic of the present invention, such that any form of association that allows for the exchange of data/instructions/operations between the microprocessor 15 and the cited elements is acceptable.

In any case, in one valid configuration, it is proposed that the microprocessor 15 be positioned outside the region delimited by the outer wall 6′, which is to say, outside the piping 6. Thus, said microprocessor 15 can be positioned, for example, in a remote center of the hospital environment.

With reference to FIG. 1, the microprocessor 15 should interpret the initial level of bioluminescence L₁ emitted by the target point T and, if said level of bioluminescence L₁ is within a predetermined range, this fact will indicate the presence of the biofilm at the target point T.

In one non-limiting example, and as previously described, the aforementioned predetermined range indicating the presence of the biofilm is delimited by wavelengths of 300 nm to 380 nm (range of action), so if the initial level of bioluminescence L₁ is within this range, this fact will indicate the presence of the biofilm.

If the presence of the biofilm has been detected, the methodology proposed in the present invention proposes the realization of the stage of eliminating the biofilm through the realization of a second light emission event P₂.

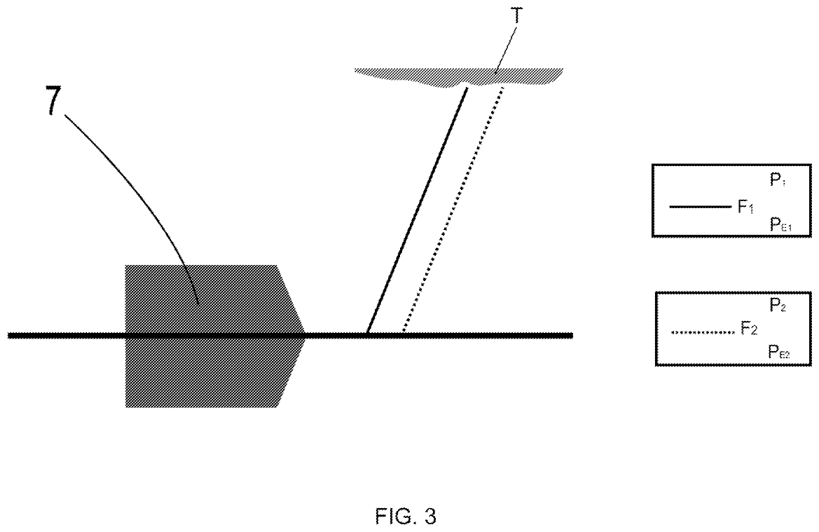

With reference to FIG. 3, the second light emission event P₂ comprises the step of emitting a second beam of light at the target point F₂, such that, in order to eliminate the biofilm, it is proposed that the second beam of light F₂ comprises an emission power P_{E2} greater than the emission power P_{E1} of the first beam of light F₁. In one modality, it is proposed that the second beam of light F₂ has the power of 10 W (a range between 8 W and 20 W is acceptable) and a wavelength of 900 nm to 1470 nm.

It is thus understood that the second beam of light F₂ is configured as a laser beam, which can be a LED beam as well as equivalents such as argon and xenon, among others. In any case, the advantage of using LED beams lies in their lower cost of acquisition.

Additionally, the present invention proposes that the second beam of light F₂ be emitted in a pulsed manner, that is, it proposes the emission of the second beam of light F₂ at each predetermined time interval. In a non-limiting manner, the second beam of light F₂ may be emitted at each 2 second interval, as shown in FIG. 4 (a).

In one equally valid mode illustrated in FIG. 4 (b), the second beam of light F₂ may be emitted continuously (constantly), i.e. uninterruptedly, for a maximum period of time, such as 10 seconds. Obviously, the reference to this time range should not be considered as a limitation of the present invention.

Furthermore, if emitted in a pulsed or continuous (constant) manner, as described above and illustrated in FIGS. 4(a) and 4(b) respectively, a purely illustrative modality of the present invention proposes that the maximum emission period of the first beam of light F₁ and the second beam of light F₂ is preferably 10 seconds, where a range of 8 s to 15 s would be acceptable. In fully valid modalities, the emission period of the first beam F₁ may be equal to or different from the emission period of the second beam F₂. It is worth noting that the values and ranges mentioned above should not be considered as limiting characteristics of the present invention.

Furthermore, the emission of the second beam of light F₂ combining the pulsed and continuous emission is also fully valid. Thus, the beam F₂ can be emitted initially in a pulsed and then continuous form, as shown in FIG. 4 (c). The reverse situation is also fully valid.

Furthermore, it is proposed that the second beam of light F₂ be emitted at the same angle α (first angle α) used in the emission of the first beam of light F₁, with reference made thereto in FIG. 2.

It is worth noting that the forms of emission illustrated in FIG. 4 for the second beam of light F₂ are also valid for the emission of the first beam of light F₁.

The teachings of the present invention also propose that during the emission of the second beam of light F₂, one should also evaluate the intensity of the bioluminescence emitted by the target point T, thus detecting a correction level of bioluminescence L₃. In this scenario, it is expected that the level of bioluminescence L₃ will be reduced throughout the application of the second beam of light F₂, thus indicating the elimination of the biofilm and also of the bacteria located therein.

Equally validly, the correction level of bioluminescence L₃ may be detected after the emission of the second beam of light F₂. Thus, after the emission of the second beam F₂, the correction level of bioluminescence L₃ is expected to have a value lower than the initial level of bioluminescence L₁ of the biofilm.

More specifically, the correction level of bioluminescence L₃ is expected to be outside the bioluminescence range indicative of the presence of biofilm, a range previously indicated as 300 nm to 380 nm.

It is understood that the present invention proposes the detection and elimination of the biofilm respectively through the emission of a first beam of light F₁ and a second beam of light F₂, where the emission power of the first beam of light F₁ is less than the emission power of the second beam of light F₂, so that the second beam of light F₂ is configured as a laser type beam (such as the LED base) with a wavelength in the range of 900 nm to 1470 nm.

In other words, the emission of the first beam of light F₁ acts as a verification step to evaluate, through the behavior of the target point T, the existence of the microorganism (biofilm) in the external wall 6′ of the pipe.

The emission of the second beam of light F₂ aims to effectively eliminate the biofilm that is at the target point T. For this reason, its emission power P_{E2} must be greater than the emission power P_{E1}.

Additionally, the detection and elimination of the biofilm occurs by evaluating the initial bioluminescence level L₁ as well as the correction bioluminescence level L₃ of the target point T.

With reference to FIGS. 1 to 4, it is worth noting that the methodology and system adopted in the present invention could easily use a greater number of light emission 4 and capture 7 elements. Thus, the use of only one fiber 4 and only one camera 5 inside a pipe 6 should not be considered as a limiting feature of the present invention.

In fully validated modalities, four fiber units 4 and four light capture elements 7 can be used, for example, to encompass a larger area of piping 6. Obviously, and depending on the area of piping 6, the use of only one fiber 4 and camera 7 would allow its entire area to be monitored.

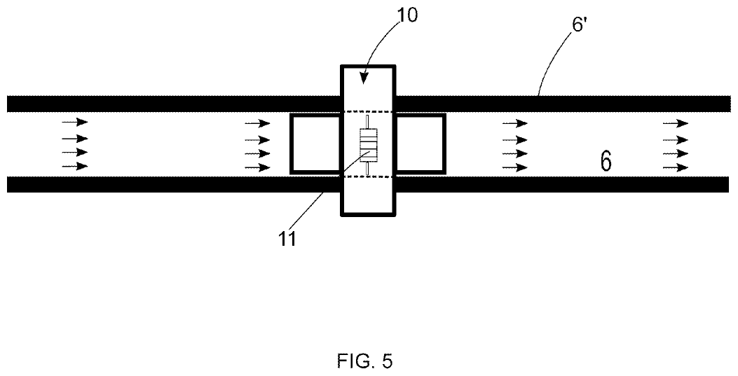

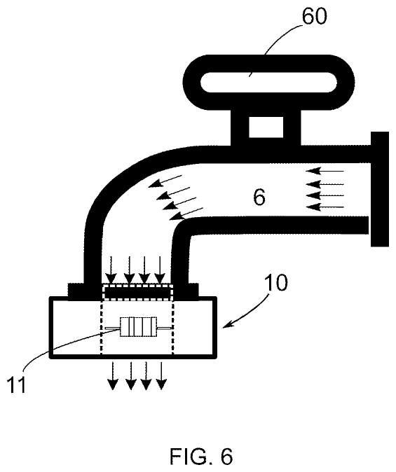

In addition to the possibility of detecting the biofilm through the positioning of the light emission element 4 at the water flow point 6, the present invention also proposes the possibility of positioning a detection module 10 at the water flow point 6, as illustrated in FIGS. 5 and 6.

This detection module 10 may be positioned, for example, inside the pipe 6, as shown in FIG. 5. Additionally, the detection module 10 can be positioned at a water outlet point, or at a tap 60, as shown in FIG. 6.

Obviously, the location of the placement of the detection module 10 as illustrated in FIGS. 5 and 6 should not be considered as a limiting feature of the present invention. In general terms, said module 10 could be positioned at any place where there is a volume of water and where it is wished to verify the possible existence of microorganisms.

The use of the detection module 10 in the field has proved to be effective in the detection of bacteria in a water flow. So, the said detection module can be used for the detection of Legionella in water pipes. Obviously, the reference to Legionella should not be considered as a limiting feature of the present invention, such that other bacteria may be detected using the methodology and system described here.

In relation to the detection module 10, this can be understood as a sensor capable of detecting the presence of microorganisms in a water flow, formed basically of a plurality of quartz crystal sensors 12, 12_{A}, 12_{B}, 12_{C}, . . . 12_{N} arranged in the form of a crystalline ring 11. The detection module 10 is capable of indicating the presence of a micro-organism by varying the oscillation frequency of the quartz sensors 12_{A}, 12_{B}, 12_{C}, . . . 12_{N}.

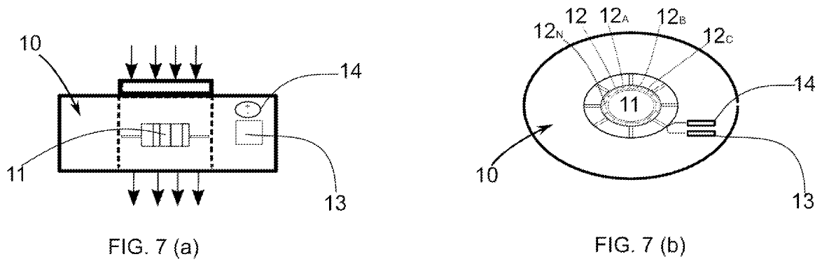

In this regard, and with reference to FIGS. 7(a) and 7 (b), the positioning of the quartz sensors 12, 12_{A}, 12_{B}, 12_{C} . . . 12_{N} is observed, thus forming the crystalline ring 11 in addition to the positioning of an electronic module 13 and battery 14 which form integral parts of the detection module 10.

The electronic module 13 has the function of applying a given oscillation frequency to the quartz sensors 12, 12_{A}, 12_{B}, 12_{C} . . . 12_{N} and also of enabling the sending of information relating to the detection of the microorganism to a remote center. Said remote center may also be associated with the microprocessor 15, as previously described. Regarding the battery 14, its function basically consists of electrically feeding the sensors 12, 12_{A}, 12_{B}, 12_{C} . . . 12_{N} and the electronic module.

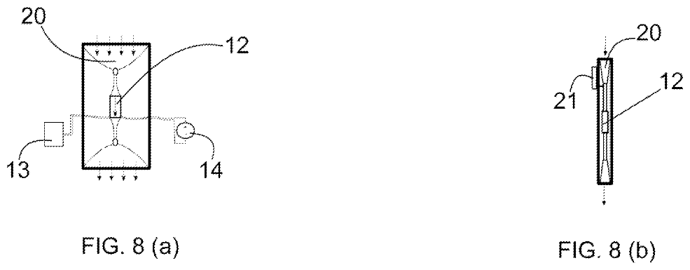

FIG. 8 illustrates an additional representation of the detection module 10, where one of the quartz sensors 12 is observed as well as the arrangement of an inlet cavity 20 for directing the water flow (which flow is indicated by means of vertical arrows) towards said sensor 12.

FIGS. 8 (a) and 8 (b) also show the previously described electronic module 13 and battery 14, as well as a binder reservoir 21 that should be associated with the sensor 12. Said binder reservoir 21 has the function of injecting said binder into the water flow, thus allowing for the analysis of the volume of water by the quartz sensor.

In this configuration, the binder is added to the water flow through the effect of the Bernoulli pressure drop (also called the Venturi effect) and due to the narrowing of the diameter of the pipe 6 through the arrangement of the detection module 10.

Specifically, the binder must bond to the microorganism, thereby increasing its mass and enabling its detection by the quartz sensors 12, 12 12_{A}, 12_{B}, 12_{C}, . . . 12_{N}. In a non-limiting description of the binders that can be used we may cite: Lectin and Lectins, among others.

More specifically, the proposed use of the detection module 10 with the plurality of quartz crystal sensors 12, 12_{A}, 12_{B}, 12_{C}, . . . 12_{N} is based on the concept of quartz crystal microbalance. In other words, the detection module 10 can be understood as a quartz crystal microbalance, as shown below.

The quartz crystal microbalance (QCM) is used to measure the mass deposited in the electrodes (sensors 12, 12_{A}, 12_{B}, 12_{C}, . . . 12_{N}) by measuring the frequency variation. The working principle of QCM is related to the piezoelectric effect. This effect is due to the property of certain materials to generate an electric field when subjected to deformations, external pressures or mass addition.

Variations in frequency corresponding to a mass addition or subtraction can be described using the Sauerbrey equation, given by the following equation:

$$Δ f = - \frac{( 2 . {f}_{0}^{2} )}{A \sqrt{{μ}_{c} {ρ}_{c}}} . Δ m$$

In this equation, Δf represents the resonance frequency variation in Hz, A is the piezoelectrically active geometric area in cm², f₀ is the resonance frequency of the crystal in Hz, ρ_{c} is the crystal density in g/cm³, μ_{c} is the shear module of the quartz crystal in g·cm⁻¹·s⁻² and Δm the mass variation in g.

However, the Sauerbrey equation was developed for use in oscillatory systems in the air and is applied only to rigid masses applied to the crystal. In the case of application in liquid media, which is the proposal of the present invention, where the viscosity of the liquid is much greater than the air, the equation that governs this behavior of mass addition in liquids has been modified (Kanazawa, K. Keiji; Gordon II, Joseph G. (July 1985). “Frequency of a quartz microbalance in contact with liquid”. Analytical Chemistry. 57 (8): 1770-1771). The equation developed by Kanazawa et al is:

Δηₗηₗf=−f₀^{3/2}(ηₗρₗ/πρ_{c}μc),

In the equation immediately above, ηₗ is the viscosity of the liquid in g·cm⁻¹·s⁻¹.

When a mass is added to the electrode surface of the quartz crystal microbalance (sensors 12, 12_{A}, 12_{B}, 12_{C}, . . . 12_{N}), there is a change in the oscillation frequency of the system and the resonance frequencies change according to the mass added to the electrodes. The fractional frequency change (Δf/f) is equal to the mass ratio added to the mass of the quartz crystal oscillator.

High frequencies in quartz crystal oscillation are necessary to obtain quantitative analyses. The viscosity effect changes the resonance frequency and the added mass effect. However, this viscosity effect becomes negligible at high frequencies. The frequency normally used by quartz crystal oscillators is between 16 MHz and 27 MHz. Other technologies such as the wireless sensor for detecting the resonance frequency of the crystal can achieve higher resonance frequencies, reaching 180 MHz, this is because in wireless sensors the excitation of the quartz crystal for the capture of the resonance frequency of the quartz crystal is performed by a pair of antennas (one antenna to excite and another to capture), without the need for a wire connected to the crystal, thus decreasing the aggregate mass and increasing the working resonance frequency of the quartz crystal. The present invention allows for the use of quartz crystal sensors that can be either wired or wireless.

When the binder element is deposited on the surface of the quartz crystal electrode, the electronic oscillator circuit (electronic module 13) of the quartz crystal microbalance, that will be applying a frequency scan, for example, every 5 seconds, which scan for example is between 0 MHz to 27 MHz or 0 MHz to 180 MHz (in the case of the wireless crystal frequency sensor), will be able to detect this fact.

More specifically, the frequency resonance peaks of the crystal electrode with the added mass of the binder (e.g. the sensor 12_{A}) will change in relation to the electrode without mass (e.g. the sensor 12_{B}). These resonance peaks are characteristic of each type of binder, which is to say, they are also characteristics of each type of microorganism that binds to the binder, and as a result it is possible to determine precisely whether there was a change in the mass of the sensor by changing the frequency peaks characteristic of a given binder used. If the existence of the microorganism has been detected by the module 10, the elimination of the microorganism in question can be carried out, using, for example, the methodology that uses the light emission 4 and capture 7 element.

Other binder elements may be used, such as enzymes or antibodies, and the characteristics of each type of binder and bacterium (microorganism) can be determined a priori and through an internal database stored in the internal memory of a microprocessor (such as the microprocessor 15 or an independent microprocessor for the detection module 10) coupled to the microbalance that will process and analyze the data derived from the detected resonance frequencies and correlate these values with the type of binder being used for the detection of the microorganism.

Thus, and through the arrangement of the light emission 4 and capture 5 elements, as well as through the possibility of using the detection module 10, the present invention also provides a system for the detection and elimination of microorganisms in a water flow.

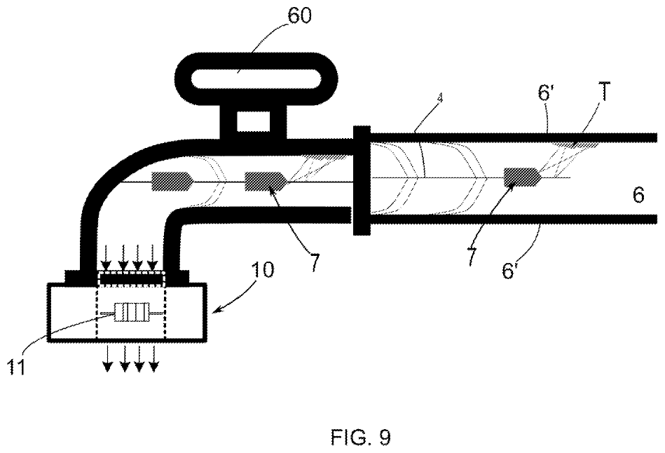

Said system may comprise the following settings: a first configuration that uses the emission 4 and capture 7 elements in isolation, as shown in FIG. 1, a second configuration that uses the detection module 10 (quartz sensor) in isolation (FIGS. 5 and 6), as well as a third configuration that uses the emission 4 and capture 7 elements together with the detection module 10, as shown in FIG. 9).

Thus, in the system shown in FIG. 9 it is understood that the detection module 10 is associated with the light emission element 4, thus providing a system capable of detecting and eliminating microorganisms in a water flow.

Thus, and using the light capture element 4 in isolation or in conjunction with the detection module 10, it is possible to monitor a given water flow and evaluate whether it contains microorganisms.

The teachings of the present invention enable the methodology and systems described to be used in a preventive manner, which is to say, with the positioning of the light capture element 4 and/or the detection module 10 inside a pipe, the existence of a microorganism can be constantly evaluated.

More specifically, the management of a hospital environment may position the light capture element 4 and/or detection module 10 in a region of interest and thus evaluate, at the desired time, whether this point of interest contains the microorganism.

It is also worth noting that it is not necessary to remove the light capture element 4 and/or the detection module 10 from the water flow after the use thereof, so these elements can be permanently positioned and thus evaluate the region of interest. In one comparison, the teachings of the present invention act as a camera monitoring circuit commonly used in public environments.

As we know, such camera circuits are able to operate 24 hours a day, thus detecting all the movement in an environment. Similarly, the teachings of the present invention act as a circuit for monitoring a water flow, which is also capable of operating 24 hours a day, if it is of interest to the user.

Furthermore, when the presence of a microorganism is detected, such an event can be stored in a database, thus indicating (in an electronic device, such as a mobile phone, computer or related equipment) date/time data regarding when the microorganism was detected as well as indicating the place where it was detected. Thus, a history relating to the detection of microorganisms can be constructed. Furthermore, if the microorganism has been detected, its elimination can be carried out, using, for example, the methodology that uses light emission 4 and capture 7 elements.

Additionally, such date/time and location information can be stored and used by the management of the hospital environment for future evaluation of the existence of new microorganisms at the same point.

It is also proposed that warning information, such as luminous or vibratory information, can be issued to the hospital management if the microorganism has been detected. In one valid modality, the warning information can be emitted on the electronic device (mobile phone, computer, tablet or related equipment) of a user or even in a hospital control room.

Moreover, it should be noted that the teachings of the present invention allow for the use of the light emission element 4 in isolation as well as in conjunction with the detection module. Furthermore, the use of only the detection module is also fully acceptable.

It is also worth noting that the reference to the range of values produced throughout this invention should obviously consider the minimum and maximum limits of the ranges of values produced as well as any value between such minimum and maximum limits. For example, the reference to a range between 300 nm and 380 comprises the limits 300 nm and 380 nm as well as any value between such values.

Finally, use of the light capture element 4 can be made without the need to interrupt the water flow of a given pipe. Obviously, its use with an interrupted water flow is also fully acceptable.

Having described an example of the preferred embodiment, it should be understood that the scope of the present invention encompasses other possible variations, being limited only by the content of the attached claims, including the possible equivalents.

What is claimed is:

1. A system for the detection and elimination of microorganisms in a water flow, the system comprising:
at least one light emission element arranged at a point in the water flow,
at least one light-capturing element at the point of the water flow,
wherein the system is configured to detect the presence of the microorganism from the first light emission event, and
wherein the system is further configured to eliminate the microorganism through the realization of a second light emission event.

2. The system according to claim 1, wherein the system also comprises a microprocessor associated with the light emission element and the light capture element, and wherein:
the light emission element is configured to emit a first beam of light at a target point and,
the microprocessor is configured to evaluate the behavior of the target point in response to the first beam of light emitted and, based on the evaluation of the behavior of the target point, the microprocessor is configured to detect the presence of the microorganism, so that,
the light emission element is configured to emit a second beam of light at the target point if the microorganism has been detected.

3. The system according to claim 2, wherein the light capture element is configured to measure an initial bioluminescence level and a final bioluminescence level of the target point so that:
the initial bioluminescence level is measured at a moment after the emission of the first beam of light, and
the final bioluminescence level is measured at a moment after the emission of the second beam of light.

4. The system according to claim 3, wherein the system also comprises a detection module arranged at the water flow point where the detection module comprises at least one crystalline ring, which crystalline ring describes a path for passage of the water flow, where the crystalline ring comprises at least one quartz crystal sensor.
